(19) United States
(12) Patent Application Publication
(10) Pub. No.: US 2025/0314583 A1
(43) Pub. Date: Oct. 9, 2025

(54) SYSTEMS AND METHODS FOR DETERMINING ANALYTE CONCENTRATION IN AIR OVER AN AGRICULTURAL FIELD

(71) Applicants: EcoFluxNet, Inc., Encinitas, CA (US); The Trustees of Princeton University, Princeton, NJ (US)

(72) Inventors: Mark Zondlo, Princeton, NJ (US); Paul Zorner, Encinitas, CA (US); Josh Collins, Princeton, NJ (US); Paul Guiguizian, Princeton, NJ (US); Anthony F. Michaels, Palos Verdes Estates, CA (US); Vladislav Sevostianov, Princeton, NJ (US); Howard Bell, Princeton, NJ (US)

(21) Appl. No.: 19/174,719

(22) Filed: Apr. 9, 2025

Related U.S. Application Data
(60) Provisional application No. 63/631,897, filed on Apr. 9, 2024.

(51) Int. Cl. G01N 21/3504; G01N 1/22; G01N 1/24

(52) U.S. Cl. CPC G01N 21/3504; G01N 1/2273; G01N 1/24; G01N 2201/06113; G01N 2201/0636

(57) ABSTRACT

Systems and methods are provided for measuring analytes (e.g., greenhouse gases) in the air over a field in an agricultural setting. The existing equipment of a center pivot and an attached arm can be used by adding reflectors and/or point sensors on the arm and an analyzer unit on the center pivot. The analyte concentration in the air can be detected down to a sensitivity of 1 part per billion or less.

### CROSS-REFERENCE TO RELATED APPLICATION

This application claims the benefit of U.S. Provisional Application Ser. No. 63/631,897, filed Apr. 9, 2024, the disclosure of which is hereby incorporated by reference in its entirety, including all figures, tables, and drawings.

### BACKGROUND

In agriculture, many greenhouse gases and air pollutants are generated and degrade the environment. Thus, limits may be placed by government on the amount of emissions that a particular farm or field may produce before fines, mitigation efforts, or specific reporting requirements can be implemented. If the actual generation of a particular gas is below the limit, the difference can be sold in a carbon credit market, touted as a sustainability metric, or marketed as a form of environmental stewardship, all of which may generate income for the owner of the farm or field. In addition, scientific researchers also want to quantify emissions of these gases to help understand how operator practices, meteorological conditions, and other interventions may increase or decrease such emissions. It is therefore important to accurately measure greenhouse gas and air pollutant emissions from agricultural activities.

### BRIEF SUMMARY

Embodiments of the subject invention provide novel and advantageous systems and methods for measuring analytes (e.g., greenhouse gases) in the air over a field in an agricultural setting. The existing equipment of a center pivot and an attached arm can be used by adding reflectors and/or point sensors on the arm and an analyzer unit on the center pivot. The analyte concentration in the air can be detected down to a sensitivity of 1 part per billion (ppb) or less.

In an embodiment, a system for measuring at least one analyte in air over an agricultural field can comprise: sensing equipment disposed on a center pivot and an arm attached to the center pivot; and an analyzer unit in operable communication with the sensing equipment. The system does not require any infrastructure to be added to the center pivot or the arm. The system can further comprise a meteorology station in operable communication with the analyzer unit, and the meteorological station can be configured to obtain meteorological data of the air over the agricultural field, air adjacent to the agricultural field, or both. The meteorological data can comprise wind speed, wind direction, air pressure, air temperature, and/or humidity. The meteorology station can be disposed within or adjacent to the agricultural field (e.g., on the center pivot). Each analyte of the at least one analyte can be a greenhouse gas or air pollutant (e.g., nitrous oxide (N₂O), ammonia (NH₃), methane (CH₄), carbon dioxide (CO₂), and/or ozone (O₃)). The system can be configured to measure the at least one analyte with a precision resolving about 1 part in 1000 of the ambient level of each respective gas in the atmosphere away from nearby sources (i.e., background levels). This is on the order of ppb or less (such as, for example, 100 ppb for CO₂, 0.1 ppb for N₂O, 0.1 ppb for NH₃, and 0.1 ppb for O₃). The system can be configured to measure the at least one analyte with a sensitivity/precision of 100 ppb or less, 10 ppb or less, 1 ppb or less, 0.1 ppb or less, 1 ppb, about 1 ppb, 0.1 ppb, or about 0.1 ppb). The analyzer unit can be configured to measure a concentration in the air over the agricultural field of each analyte of the at least one analyte based on spikes and valleys compared to a respective background concentration of each analyte of the at least one analyte.

In an embodiment, the sensing equipment can comprise: at least one light source (e.g., laser) disposed on the center pivot; and a plurality of reflectors disposed on the arm and configured to reflect light from the at least one light source. The plurality of reflectors can be disposed at regular intervals along the arm. The plurality of reflectors can be disposed close enough to each other along the arm for the analyzer unit to generate a map of a concentration of the at least one analyte with a predetermined desired granularity. Each light source of the at least one light source can be configured to provide light at a predetermined wavelength for a particular analyte, and the predetermined wavelength can be in the mid infrared (mid IR) range (i.e., 2 micrometers (μm) to 30 μm). The sensing equipment can further comprise a detector disposed on the center pivot and configured to receive signals of light reflected from the plurality of reflectors. The analyzer unit can comprise a computer and software stored thereon that is configured to receive the signals of light reflected from the plurality of reflectors and convert them to data indicative of a concentration of the at least one analyte in the air. The analyzer unit can convert the signals via wavelength modulation spectroscopy, direct absorption spectroscopy, or both. The data indicative of the concentration of the at least one analyte in the air can comprise at least one of: spatial information of the concentration of the at least one analyte in the air; volume information of the concentration of the at least one analyte in the air; and a flux of the concentration of the at least one analyte in the air. The system can further comprise a display in operable communication with the analyzer unit, and the analyzer unit can be configured to display the data indicative of the concentration of the at least one analyte in the air on the display. Each reflector of the plurality of reflectors can be, for example, a retroreflector configured to reflect mid-IR light. The retroreflector can comprise a base substrate and a coating layer disposed on the base substrate. The base layer can comprise a thermoplastic material (e.g., a polymer, such as polymethyl methacrylate (PMMA)). The coating layer can comprise a metal (e.g., aluminum (Al), gold (Au), silver (Ag), or a combination thereof). The coating layer can have a thickness of, for example, 10,000Angstroms or less (e.g., 5,000 Angstroms or less, such as 2,500 Angstroms or about 2,500 Angstroms). The retroreflector can further comprise: an adhesive layer disposed between the base substrate and the coating layer; and/or a protective layer disposed on the coating layer. The adhesive layer can comprise a transition metal (e.g., titanium (Ti), chromium (Cr), or a combination thereof). The adhesive layer can have a thickness of, for example, 10,000 Angstroms or less (e.g., 5,000 Angstroms or less, such as 500 Angstroms or about 500 Angstroms). The protective layer can comprise an insulative material (e.g., silicon oxide). The retroreflector can have a total thickness of 50 millimeters (mm) or less (e.g., 25 mm or less, 10 mm or less, 4 mm or less, about 4 mm, or 4 mm).

In an embodiment, the sensing equipment can comprise: a plurality of point sensors disposed on the arm and configured to suck air thereinto from the air over the agricultural field; and a main tube connected to the analyzer unit and in fluid communication with the plurality of point sensors. The analyzer unit can comprise a pumping element configured to pump air from the plurality of point sensors to the analyzer unit through the main tube. The sensing equipment can further comprise a plurality of connection tubes respectively connecting the plurality of point sensors to the main tube. The plurality of point sensors can be disposed at regular intervals along the arm. The plurality of point sensors can be disposed close enough to each other along the arm for the analyzer unit to generate a map of a concentration of the at least one analyte with a predetermined desired granularity. The pumping element can comprise a fan. The analyzer unit can comprise a computer and software stored thereon that is configured to receive the air sucked into the plurality of point sensors and convert it to data indicative of a concentration of the at least one analyte in the air. The analyzer unit can convert the air via wavelength modulation spectroscopy, direct absorption spectroscopy, or both. The data indicative of the concentration of the at least one analyte in the air can comprise at least one of: spatial information of the concentration of the at least one analyte in the air; volume information of the concentration of the at least one analyte in the air; and a flux of the concentration of the at least one analyte in the air. The system can further comprise a display in operable communication with the analyzer unit, and the analyzer unit can be configured to display the data indicative of the concentration of the at least one analyte in the air on the display. The plurality of point sensors can be disposed along the arm at a plurality of different heights, as measured from the ground of the agricultural field.

In another embodiment, a method for measuring at least one analyte in air over an agricultural field can comprise: providing a system as disclosed herein (such as one having any combination of features from the previous three paragraphs); moving the arm around the center pivot (or allowing the arm to move around the center pivot according to its normal function); using the sensing equipment to obtain air or signals of reflected light; and using the analyzer unit to convert the air or the signals of reflected light to data indicative of a concentration of the at least one analyte in the air. The method can further comprise displaying, on a display in operable communication with the analyzer unit, the data indicative of a concentration of the at least one analyte in the air.

### BRIEF DESCRIPTION OF DRAWINGS

FIGS. 1A-1B show schematic view of a center pivot system, according to an embodiment of the subject invention.

FIGS. 2A-2G show schematic views of a center pivot that can be used with a center pivot system, according to embodiments of the subject invention.

FIGS. 3A-3J show schematic views of reflectors that can be used with a center pivot system, according to embodiments of the subject invention.

FIGS. 4A-4I show schematic views of sensors that can be used with a center pivot system, according to embodiments of the subject invention.

FIGS. 5A-5H show schematic views of sprinkler drops with valves and tubing that can be used with a center pivot system, according to embodiments of the subject invention.

FIG. 6 shows an image of a portion of a center pivot and an attached arm having reflectors disposed thereon, according to an embodiment of the subject invention. The inset shows an image of an example reflector that may be used with embodiments of the subject invention.

FIG. 7 shows an example of a spatial map of analyte concentration that can obtained using systems and methods of embodiments of the subject invention.

FIG. 8 shows an image of an example reflector that may be used with embodiments of the subject invention. The scale bar is 10 millimeters (mm).

### DETAILED DESCRIPTION

Embodiments of the subject invention provide novel and advantageous systems and methods for measuring analytes (e.g., greenhouse gases) in the air over a field in an agricultural setting. The existing equipment of a center pivot and an attached arm can be used by adding reflectors and/or point sensors on the arm and an analyzer unit on the center pivot. The analyte concentration in the air can be detected down to a sensitivity of 1 part per billion (ppb) or less.

Many agricultural fields utilize a center pivot with an attached arm that can move around the center pivot to provide irrigation or other functions to the field. The arm can extend in a radial direction (of the field) from the center pivot over a portion of, most of, almost all of, or an entirety of, a radius of the field. The field can have an area of, for example, 164 acres, about 164 acres (or less depending on equipment configuration), 1 square mile, or about 1 square mile (or less depending on equipment configuration).

Embodiments of the subject invention can advantageously measure analyte concentration in the air above an agricultural field with very high sensitivity utilizing only the existing infrastructure of a center pivot and attached arm. Additional elements are added to the center pivot and arm (e.g., analyzer unit, reflectors and/or point sensors, and/or a meteorology station), but these can be disposed on and/or attached to the existing infrastructure such that no new towers or the like need to be constructed or installed at or near the field. In one embodiment, a tower can be constructed or installed for the meteorology station, but this is not required and the meteorology station can instead be disposed on and/or attached to the center pivot, the arm, or an existing fence around the field.

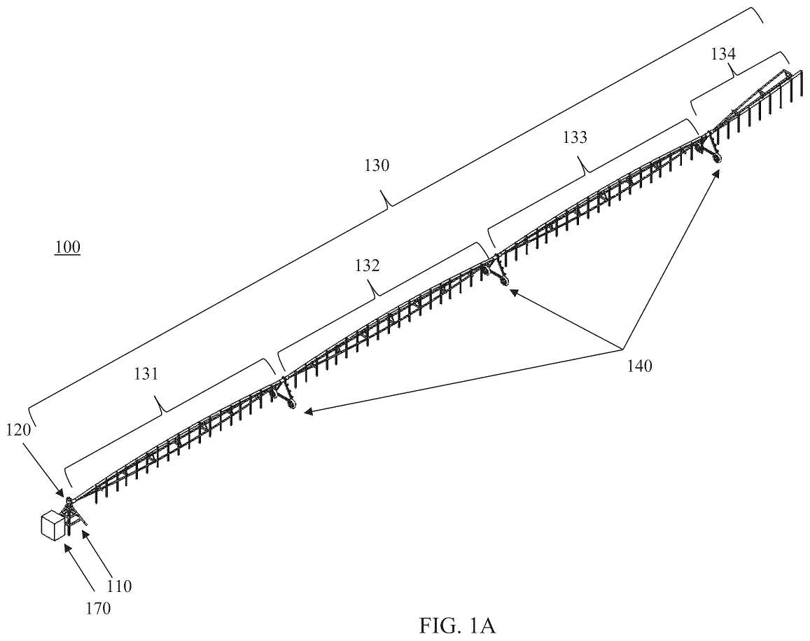

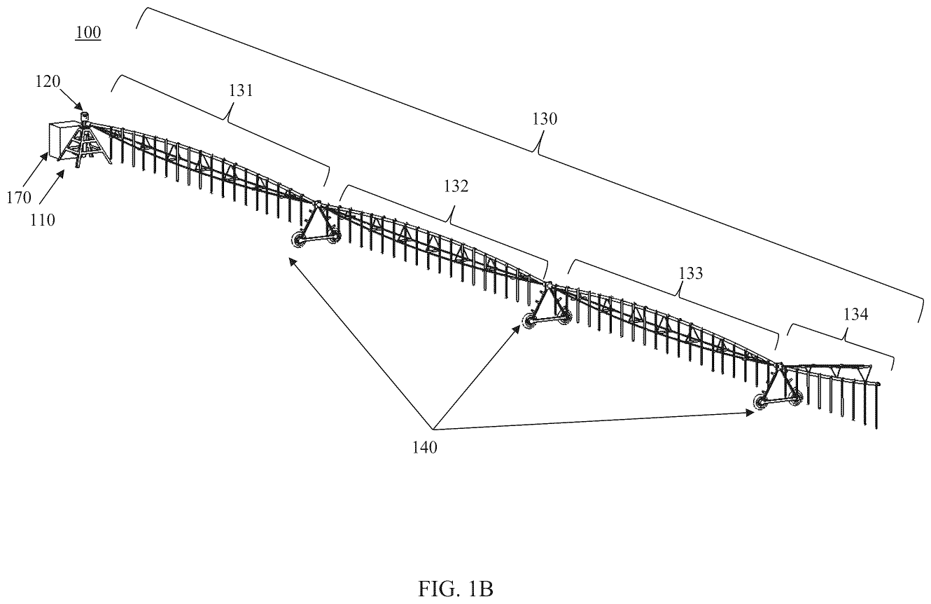

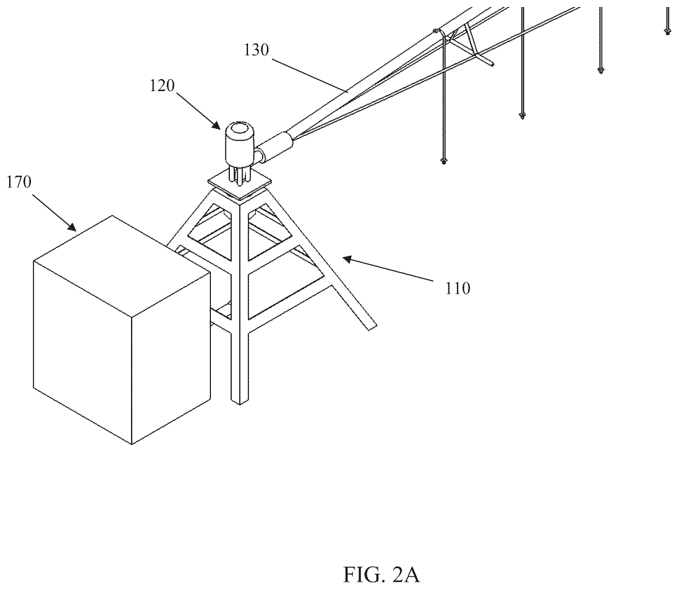

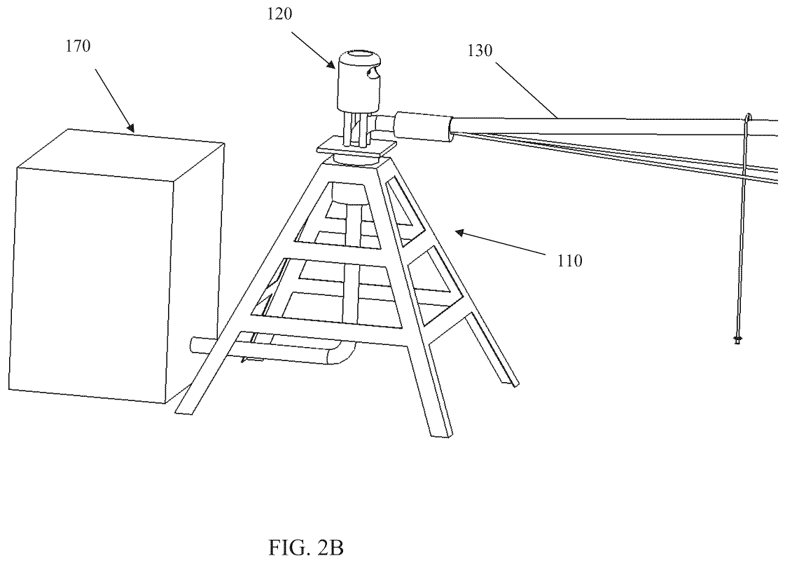

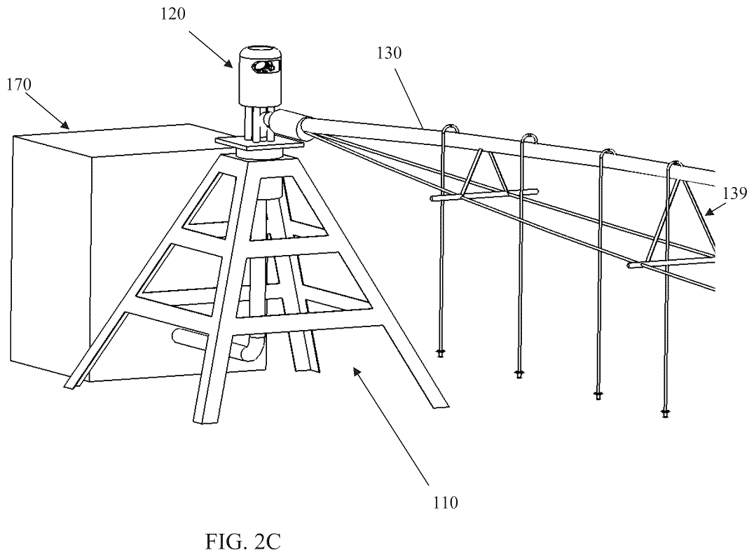

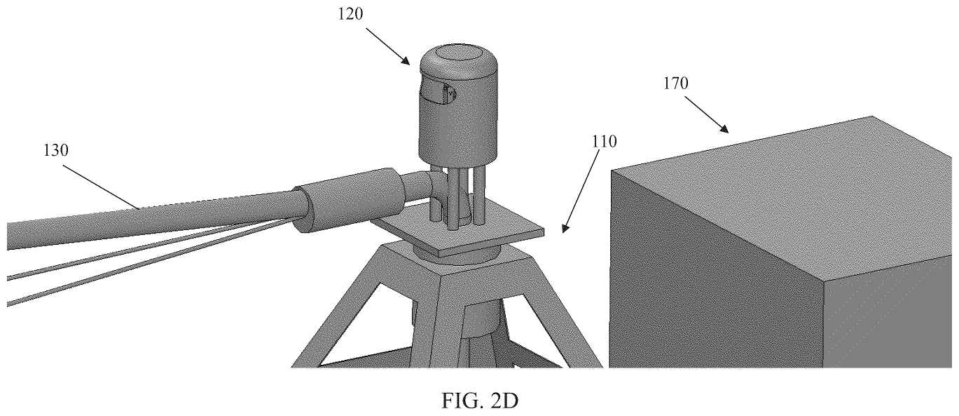

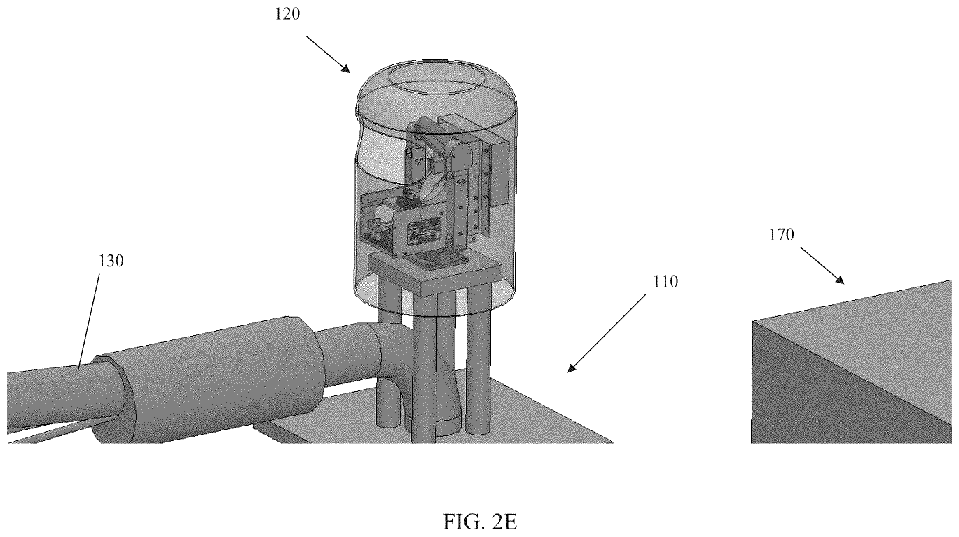

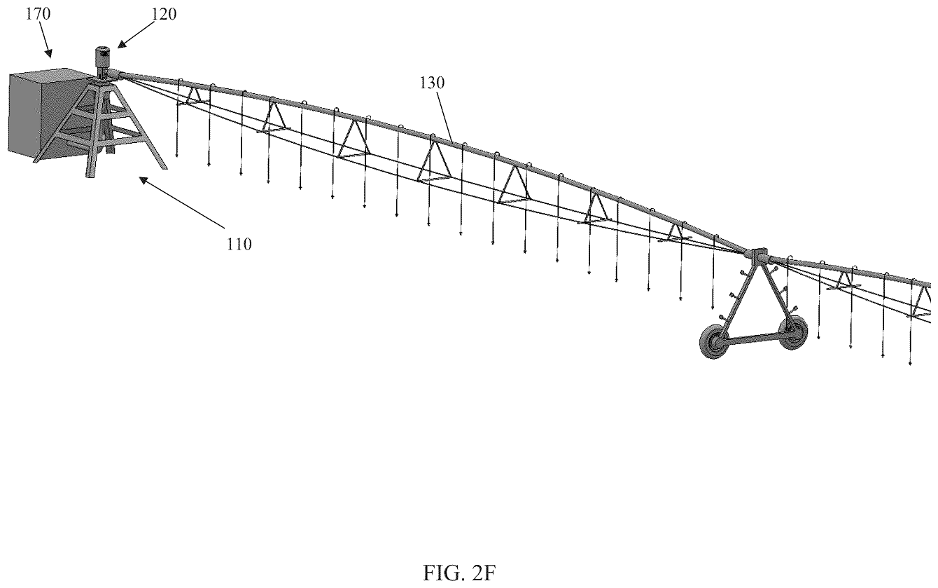

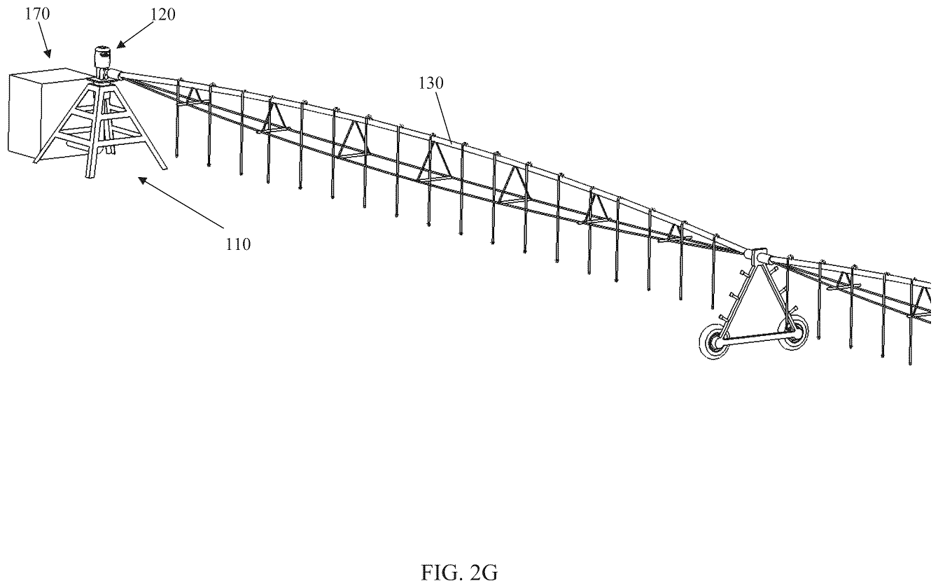

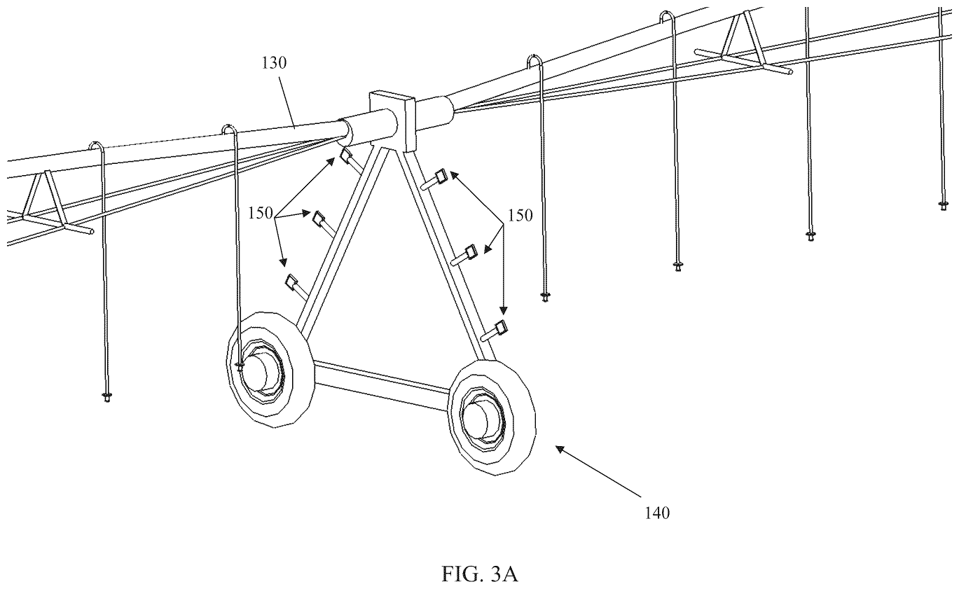

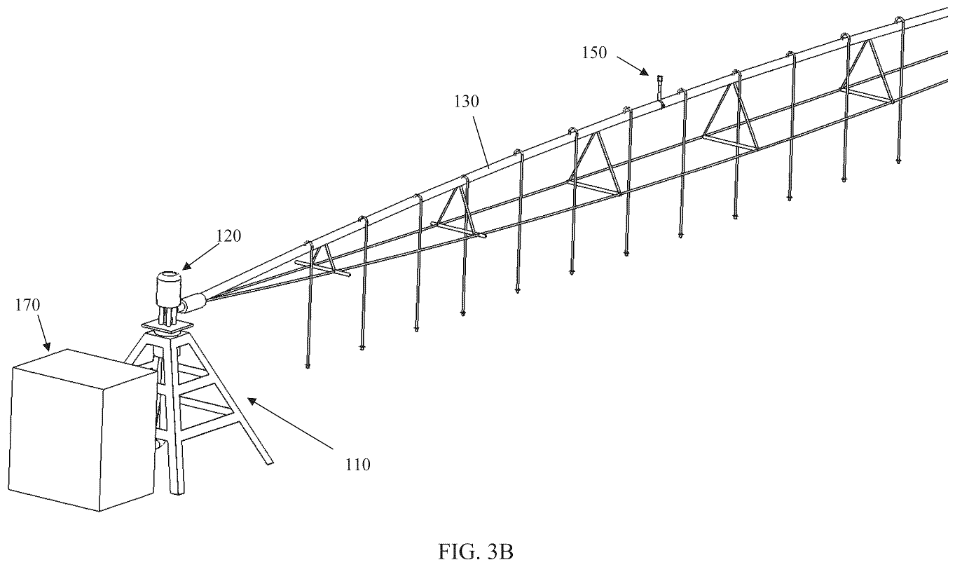

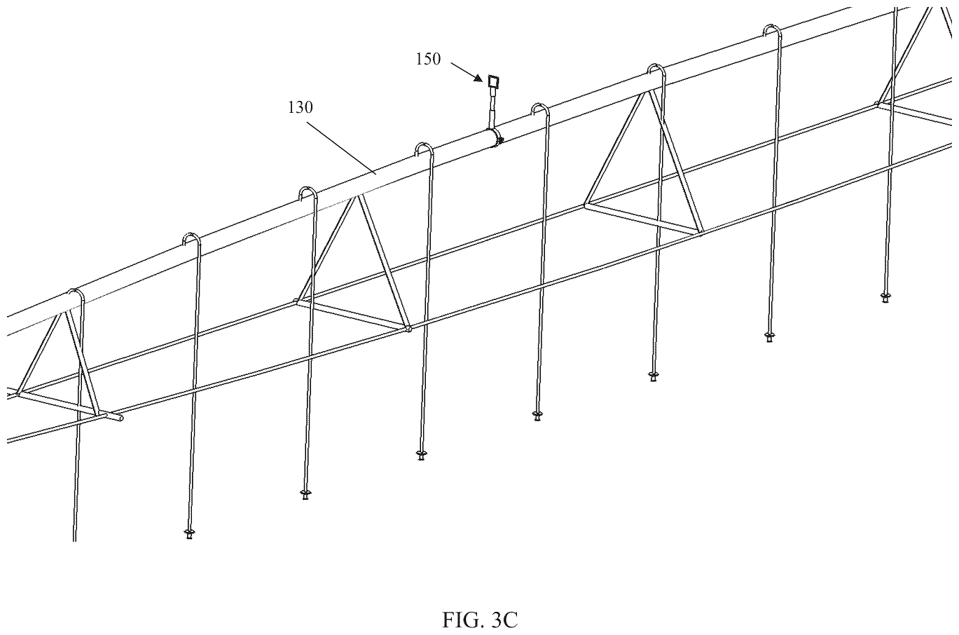

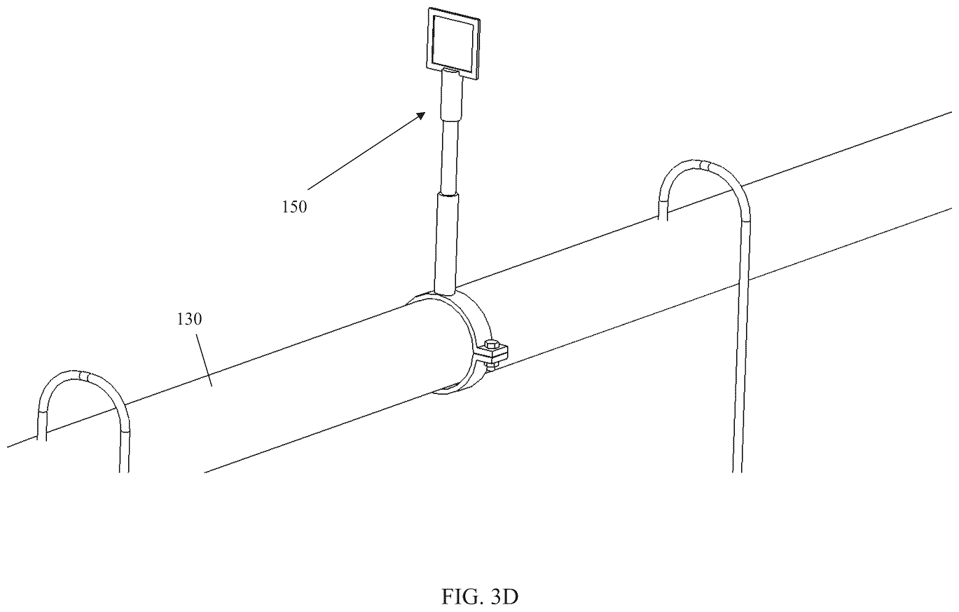

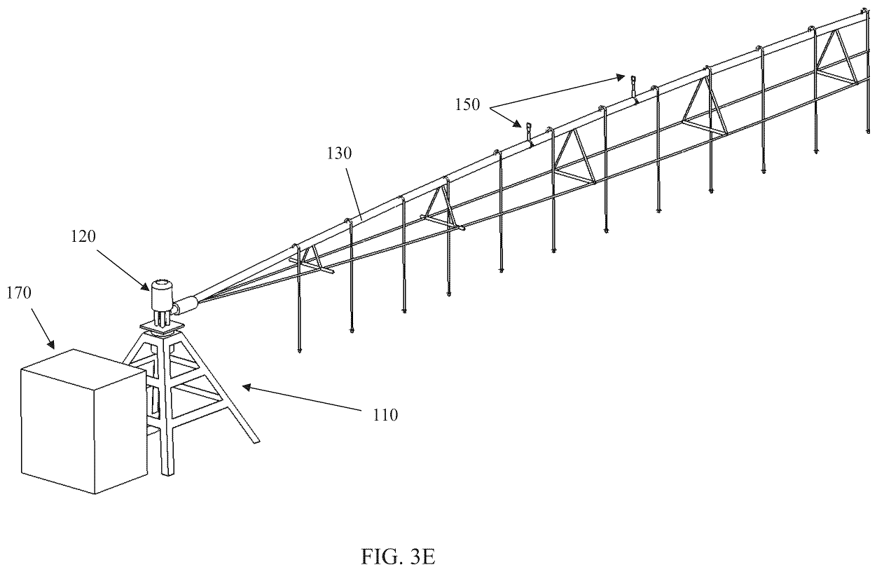

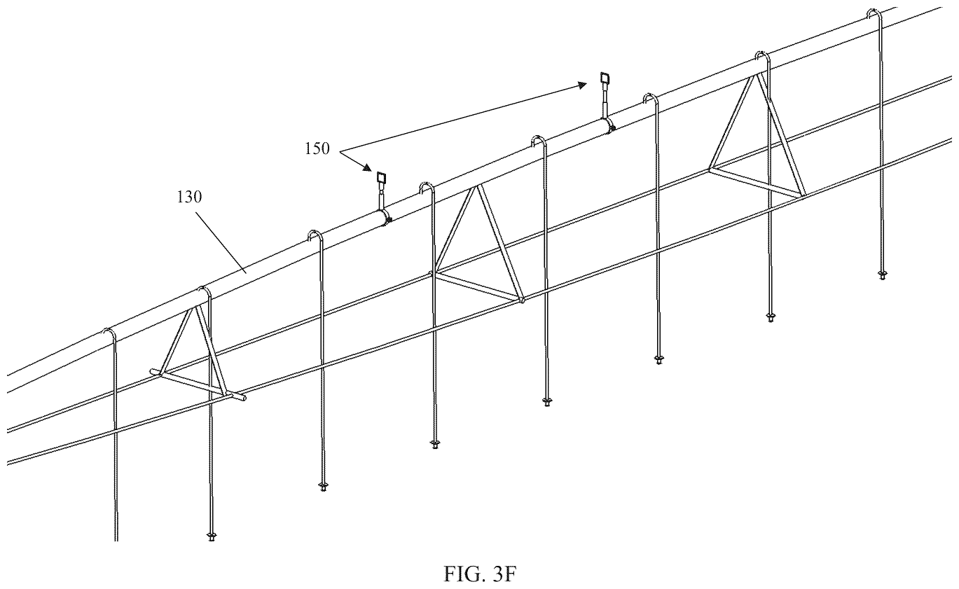

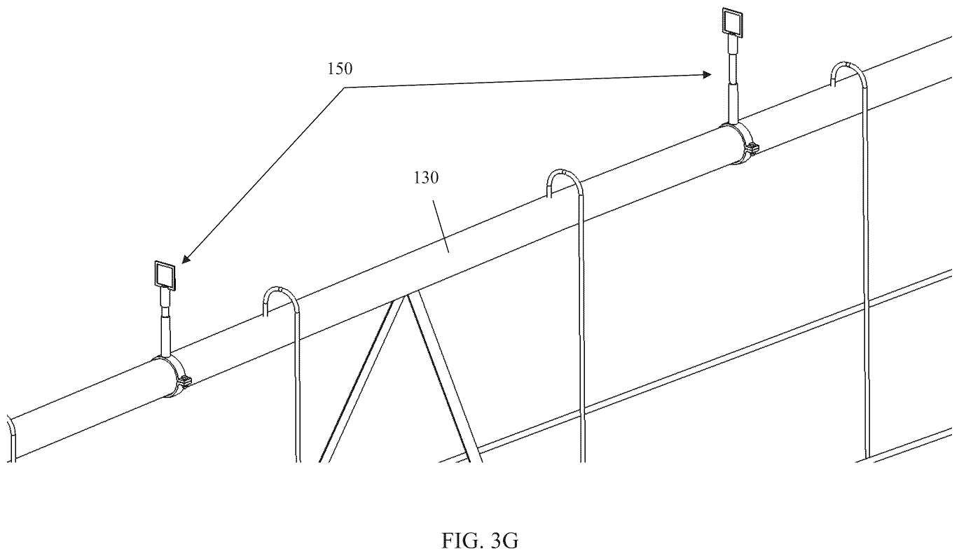

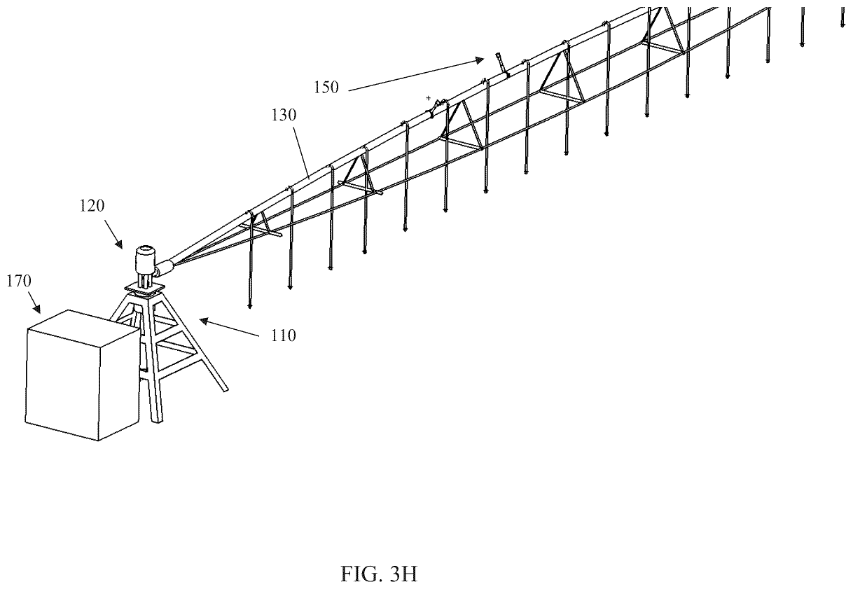

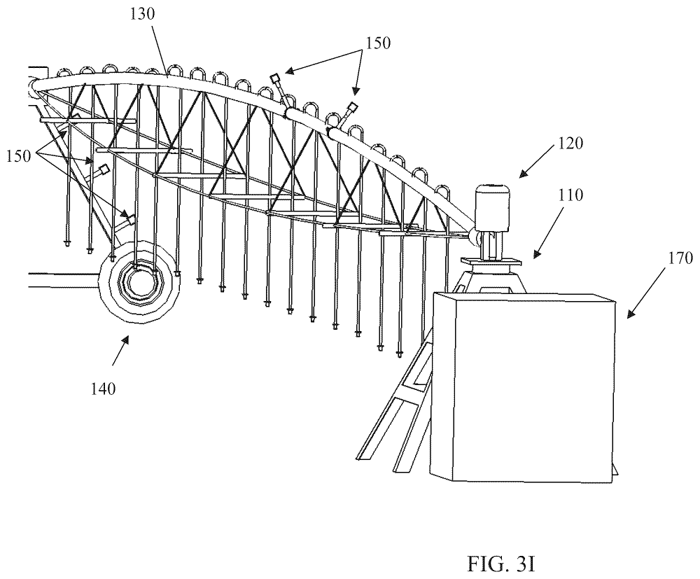

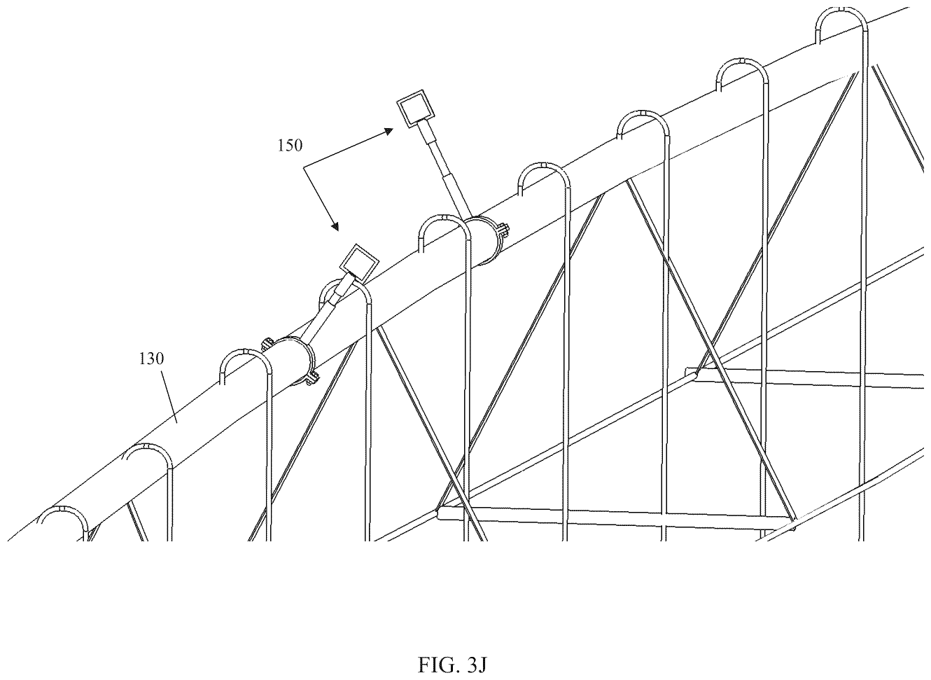

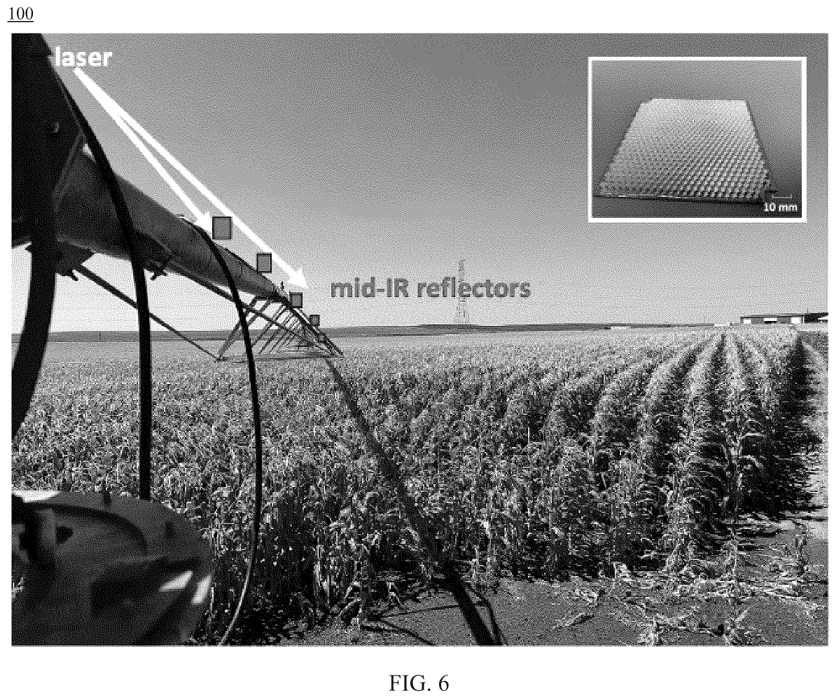

FIGS. 1A-1B show schematic view of a center pivot system, according to an embodiment of the subject invention; FIGS. 2A-2G show schematic views of a center pivot that can be used with a center pivot system, according to embodiments of the subject invention; FIGS. 3A-3J show schematic views of reflectors that can be used with a center pivot system, according to embodiments of the subject invention; and FIG. 6 shows an image of a portion of a center pivot and an attached arm having reflectors disposed thereon, according to an embodiment of the subject invention. Referring to FIGS. 1A-3J and 6, in some embodiments, at least one light source 120 (which can be referred to herein as a laser, even though it may be a light emitting diode (LED) or other light source in some embodiments) can be disposed on and/or attached to the center pivot 110, each light source 120 being configured to provide light at a respective predetermined wavelength in a direction along the arm 130 attached to the center pivot 110. A plurality of reflectors 150 can be disposed on and/or attached to the arm 130. The reflectors 150 are configured to receive light from the at least one laser 120 and reflect it back to a detector (e.g., an image sensor) disposed on and/or attached to the center pivot 110. The detector (which can be incorporated into the casing having the laser 120; see, e.g., FIG. 2E) provides signals of the reflected light to an analyzer unit 170 in operable communication with the detector and/or the at least one laser 120, and which can be disposed on and/or attached to the center pivot 110 (or the arm 130) (or can be disposed near the center pivot and/or the arm).

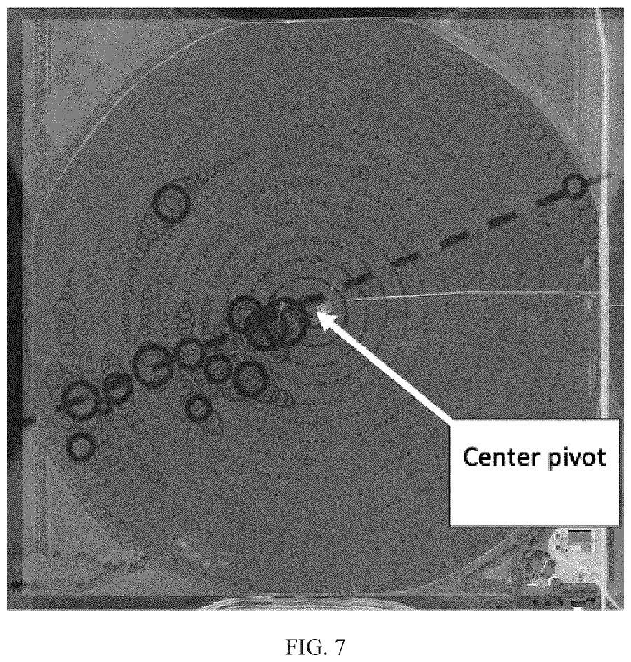

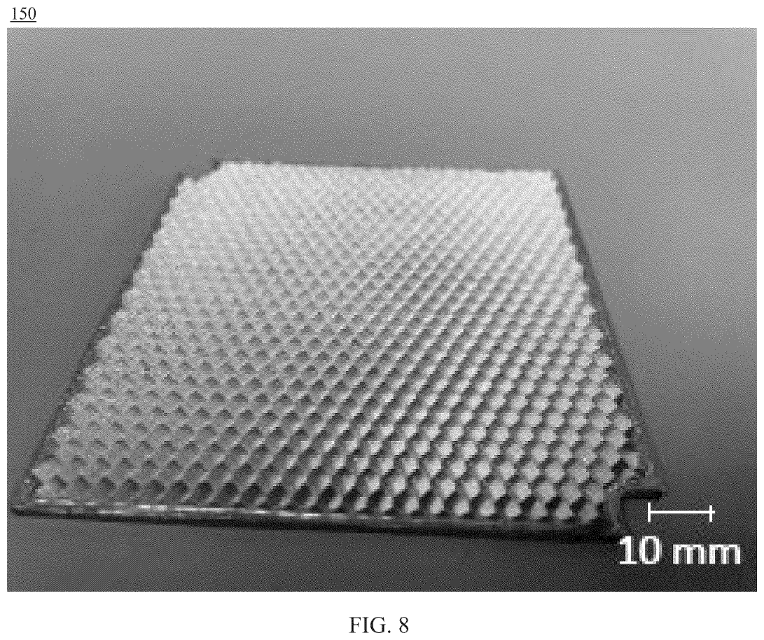

The analyzer unit can be, for example, a computer with software stored thereon that is configured to receive the signals and convert them to data indicative of the analyte concentration in the air. The optically absorbed signals can be converted via, for example, wavelength modulation spectroscopy or direct absorption spectroscopy, though embodiments are not limited thereto. The data indicative of the analyte concentration in the air can include spatial information (e.g., the concentration in space within the field) and/or volume information (e.g., the concentration spatially and by height from the ground). The data indicative of the analyte concentration can also include a flux of the analyte concentration. The analyzer unit can be in operable communication with a display on which the data indicative of the analyte concentration in the air can be displayed. The display can be located on the center pivot or the arm or can be remote from the computer, in which case the data can be transmitted to the display wirelessly or via a wire (e.g., a buried wire). As the arm 130 moves around the center pivot 110 (e.g., rolls on the wheels 140), a full picture of analyte concentration in the entire field can be obtained, as shown in FIG. 7.

Each laser can be configured to provide light at a wavelength in the mid infrared (mid IR) range (i.e., 2 micrometers (μm) to 30 μm), and at a specific wavelength for a particular analyte. That is, the at least one laser can include a first laser configured to provide light at a first mid-IR wavelength targeted to obtain the concentration of a first analyte, a second laser configured to provide light at a second mid-IR wavelength targeted to obtain the concentration of a second analyte, a third laser configured to provide light at a third mid-IR wavelength targeted to obtain the concentration of a third analyte, and/or a fourth laser configured to provide light at a fourth mid-IR wavelength targeted to obtain the concentration of a fourth analyte, etc. In the case where more than one laser is present, only one laser is operated at a time to obtain the concentration of a single analyte at a time.

The reflectors 150 can be positioned along the arm 130 in any reasonable manner. For example, a reflector 150 can be disposed on and/or attached to each segment (e.g., 131,132,133,134) of the arm 130 (or every other segment or every third segment, etc.) or each truss 139 of the arm (or every other truss or every third truss, etc.). The reflectors 150 can also be disposed in a non-regular manner, with some segments (e.g., 131,132,133,134) or trusses 139 having one or more reflectors and others having no reflectors or less reflectors. In a preferred embodiment, the reflectors 150 are disposed at regular intervals along the arm 130 and disposed close enough to each other to generate a map of the analyte concentration with a desired granularity (e.g., one reflector disposed on each segment or truss of the arm).

The reflector 150 can be any suitable reflector that reflects mid-IR laser light. In some embodiments, a new type of reflector can be used that is both cheap and effective. Each reflector can be a retroreflector configured for mid-IR light and be of the type that is otherwise only available in the related art for visible light. The retroreflector can comprise a base substrate (e.g., a thermoplastic material, such as a polymer (e.g., polymethyl methacrylate (PMMA))), an optional adhesive layer disposed on the base substrate, a coating layer disposed on the base substrate and any optional adhesive layer, and an optional protective layer disposed

JA\EFN\100X\Application\Application-asfiled.docx/on the coating layer. The coating layer can be, for example, a metal such as aluminum (Al), gold (Au), silver (Ag), or a combination thereof. The coating layer can have a thickness of, for example, 10,000 Angstroms or less (e.g., 5,000 Angstroms or less, such as 2,500 Angstroms or about 2,500 Angstroms). The optional adhesive layer can comprise, for example, a transition metal (e.g., titanium (Ti), chromium (Cr), or a combination thereof). The optional adhesive layer can have a thickness of, for example, 10,000 Angstroms or less (e.g., 5,000 Angstroms or less, such as 500 Angstroms or about 500 Angstroms). The optional protective layer can comprise, for example, an insulative material (e.g., silicon oxide). The retroreflector can have a total thickness of, for example, 50 millimeters (mm) or less (e.g., 25 mm or less, 10 mm or less, 4 mm or less, about 4 mm, or 4 mm). While related art reflectors can cost in the thousands of dollars (USD), retroreflectors of the type discussed in detail herein can be effective while only costing on the order of tens of dollars or less.

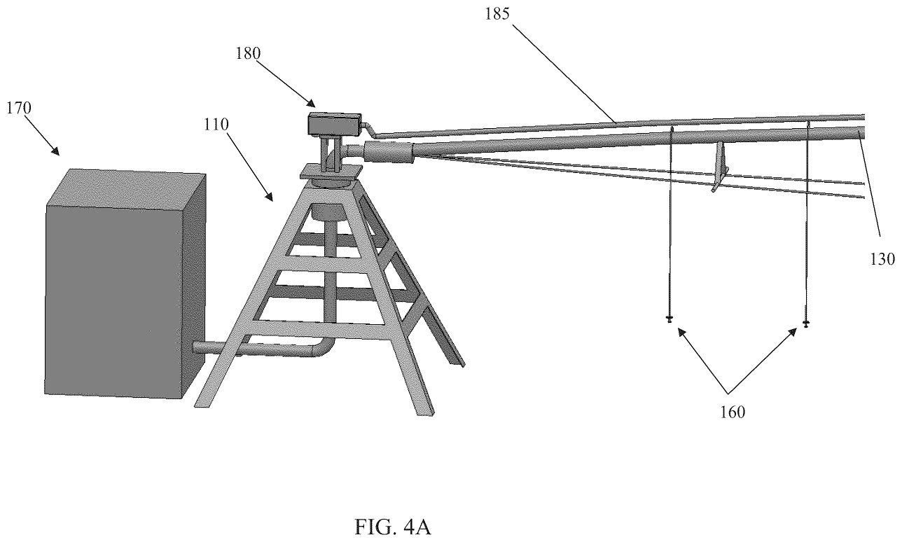

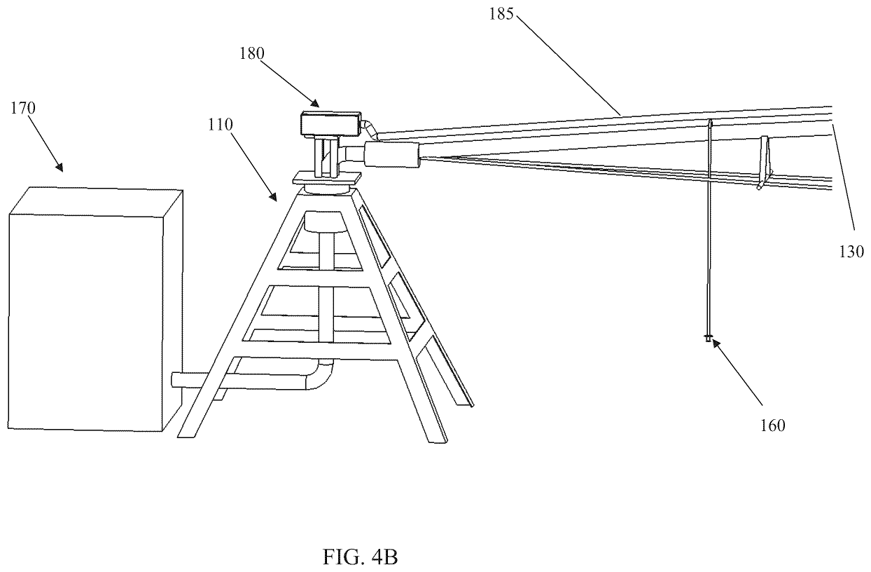

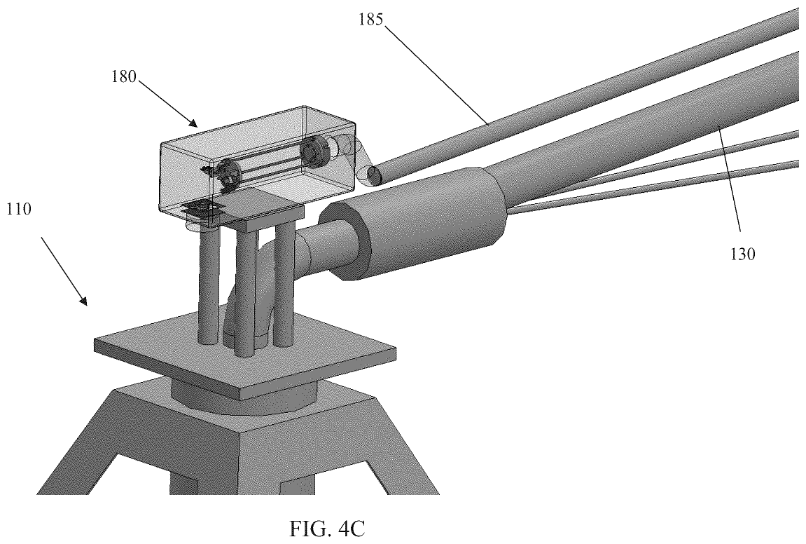

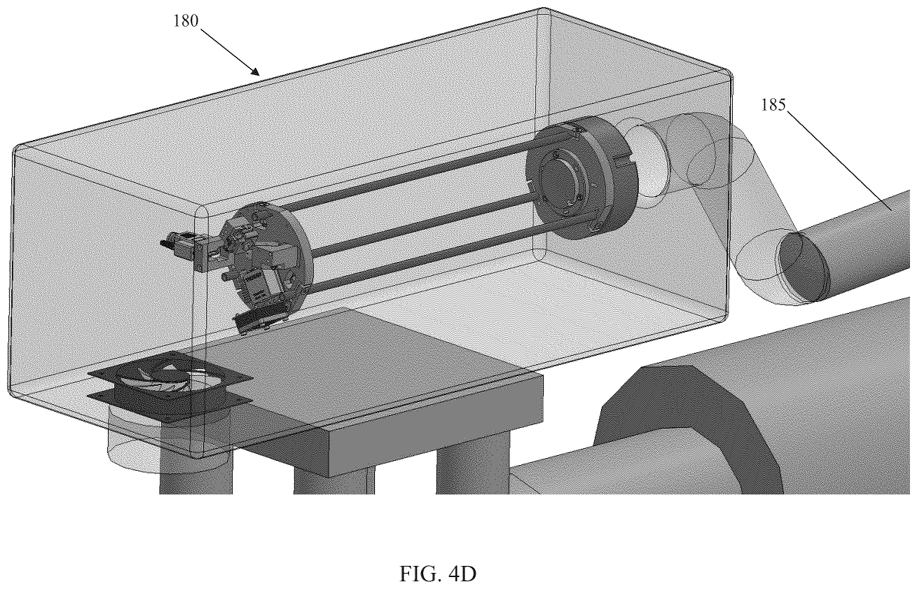

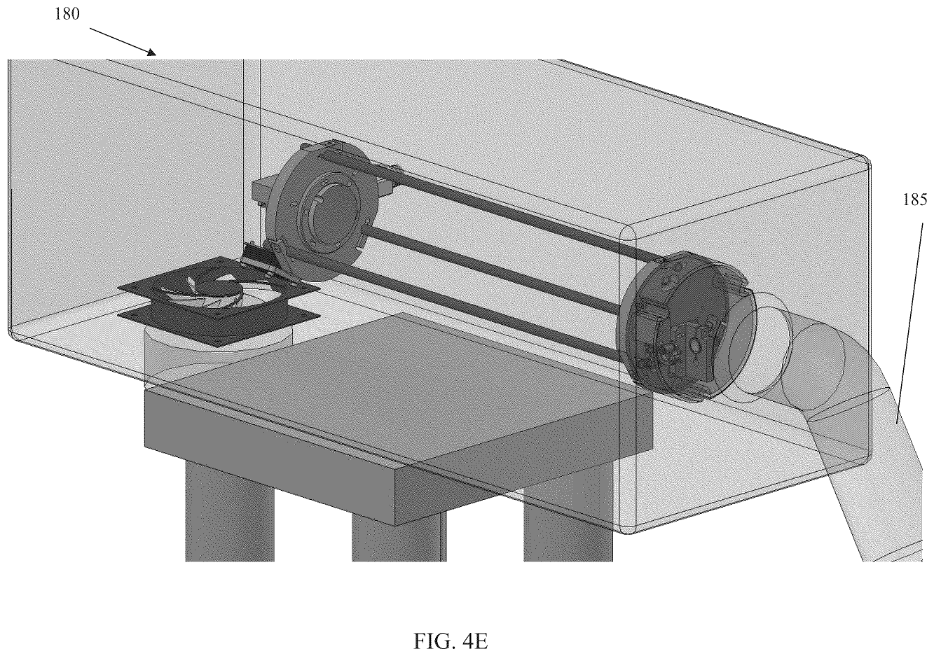

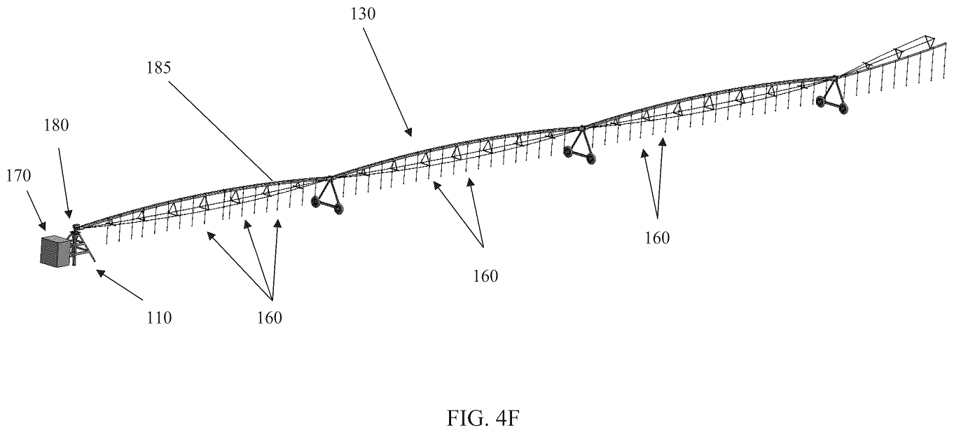

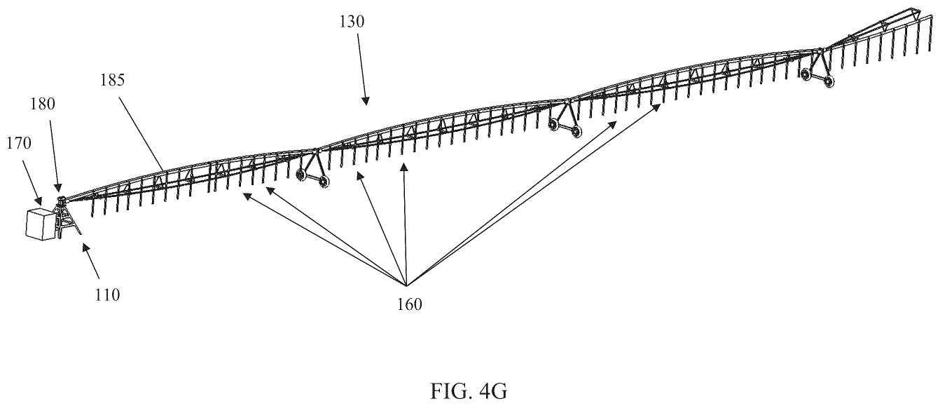

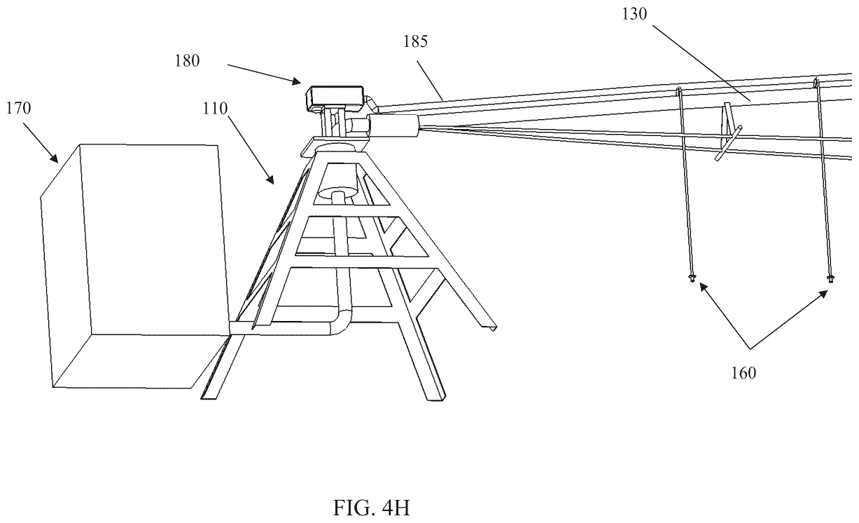

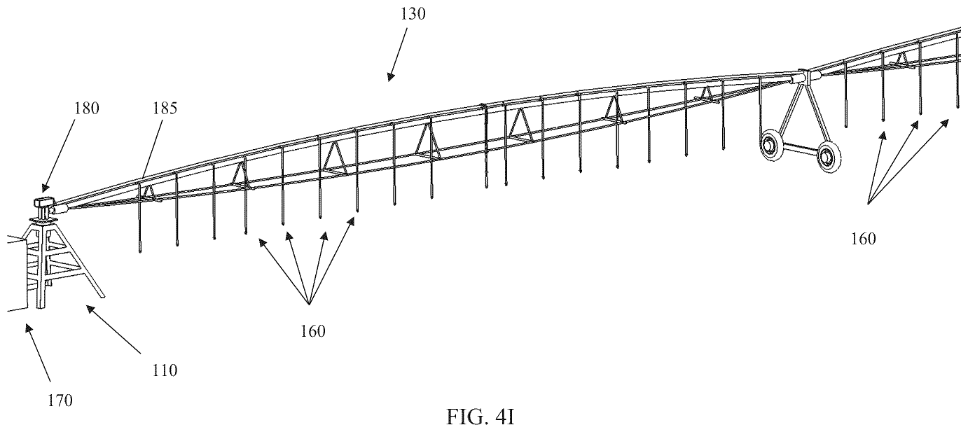

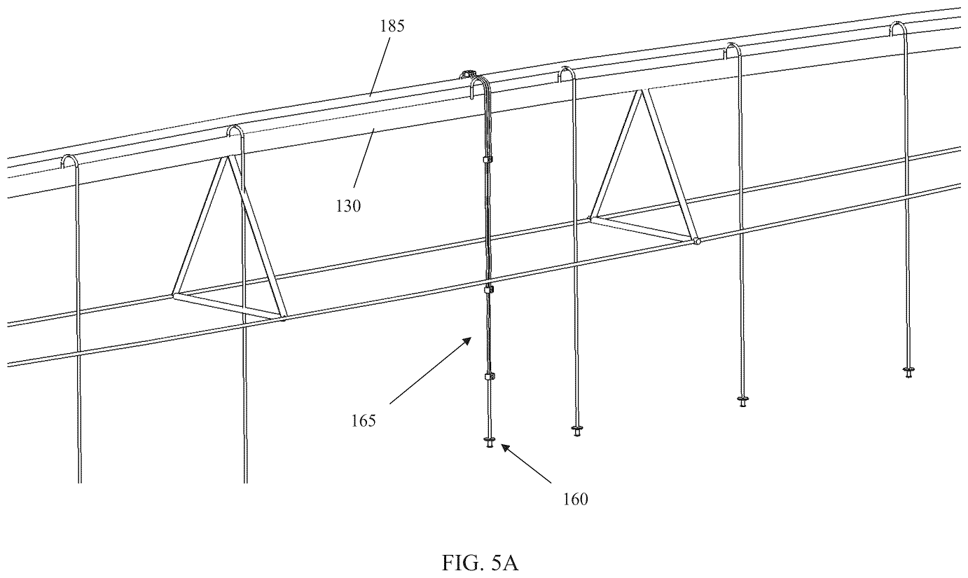

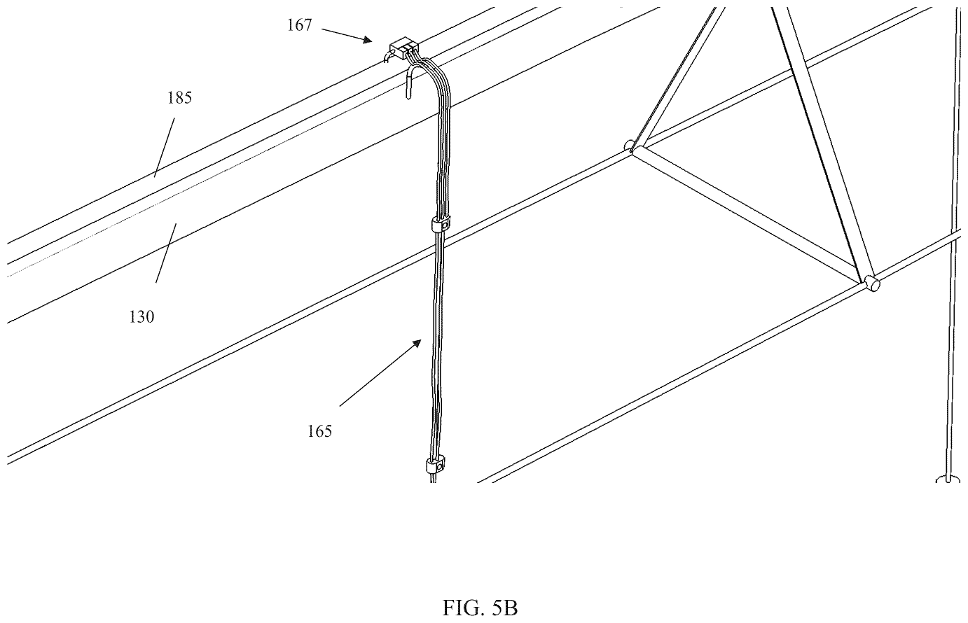

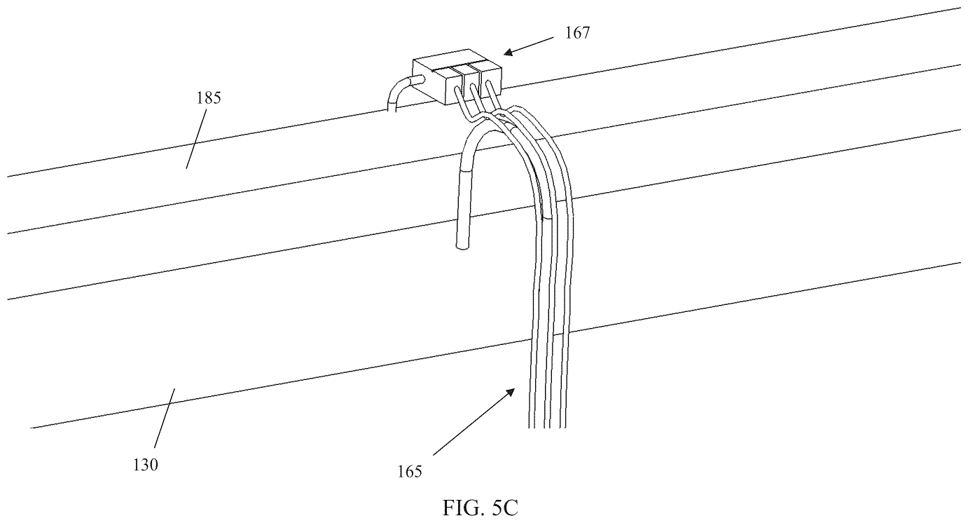

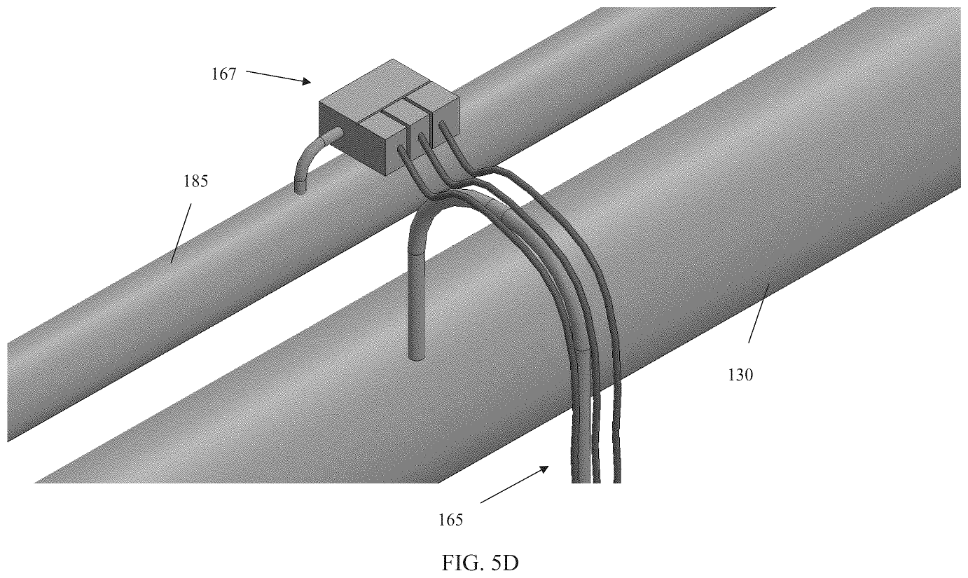

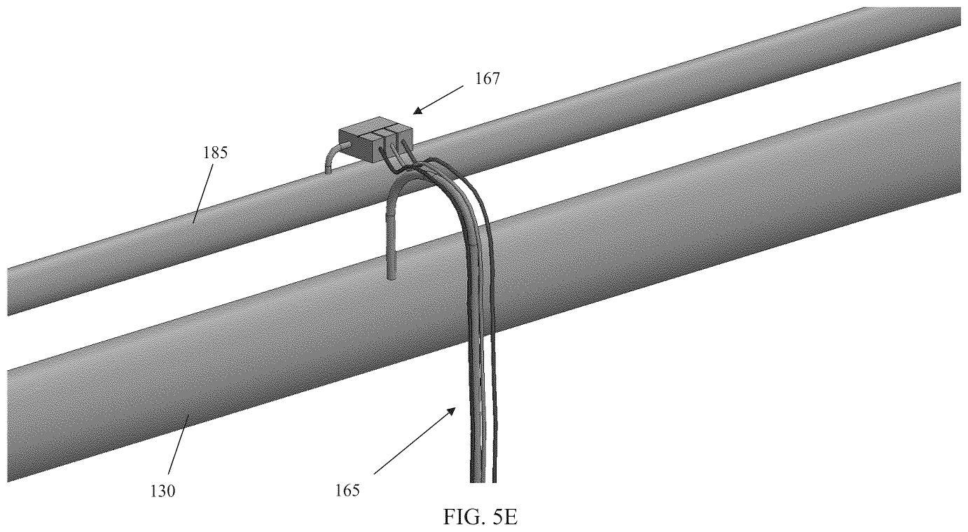

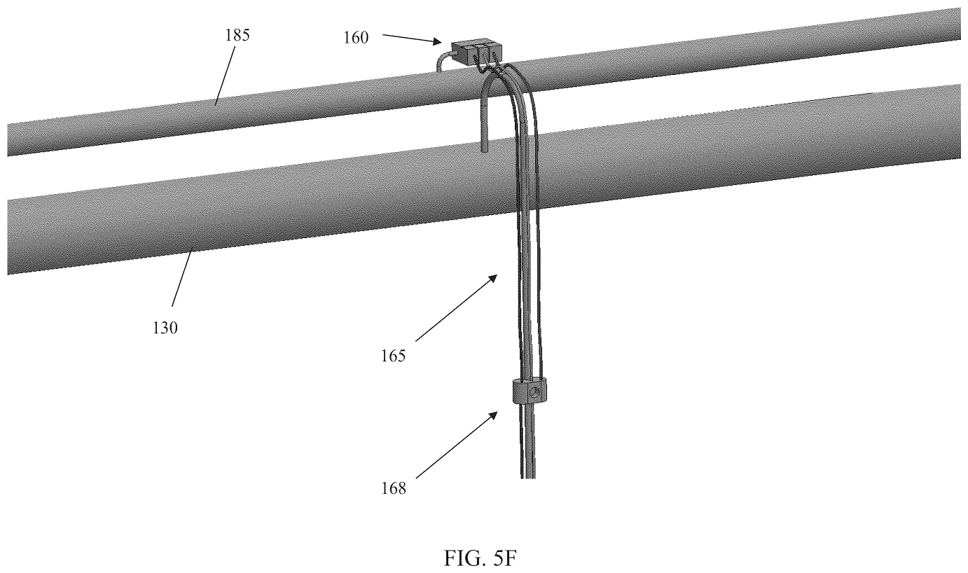

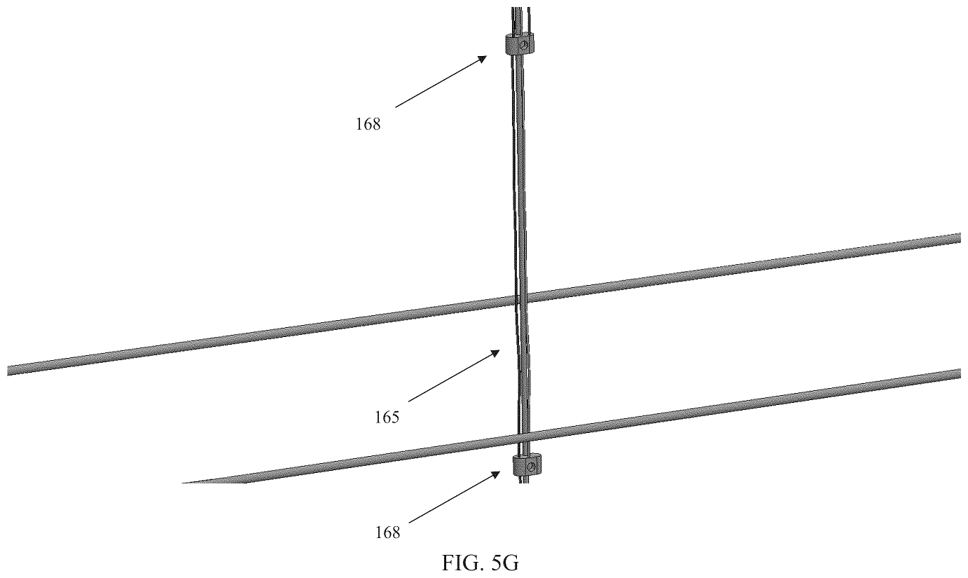

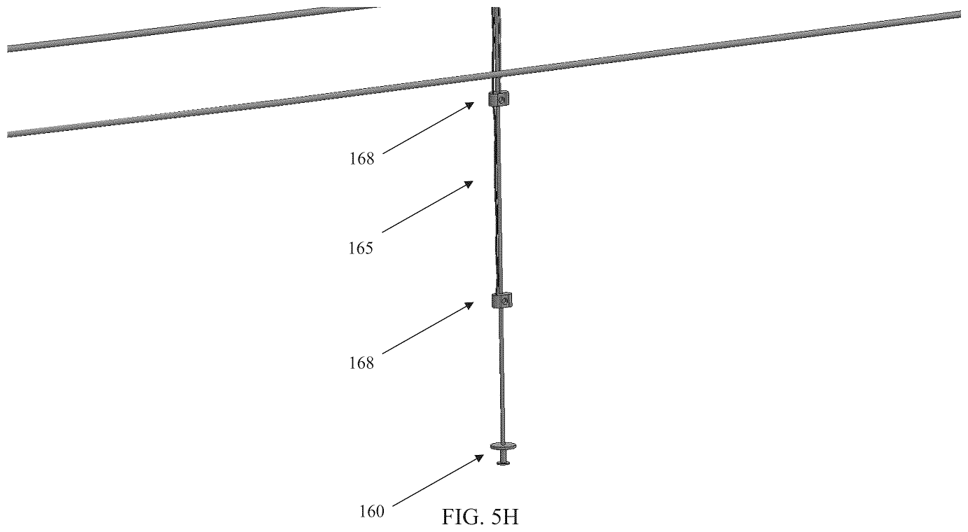

In some embodiments, point sensors can be disposed on and/or attached to the arm and can be configured to suck in air. FIGS. 4A-4I show schematic views of sensors that can be used with a center pivot system, according to embodiments of the subject invention; and FIGS. 5A-5H show schematic views of sprinkler drops with valves and tubing that can be used with a center pivot system, according to embodiments of the subject invention. Referring to FIGS. 4A-5H, an analyzer unit 170,180 can be disposed on and/or attached to the center pivot 110 (or the arm 130) (or can be disposed near the center pivot and/or the arm), and a series of tubes 165 can be respectively connected to the point sensors 160 and can all connect to the analyzer unit 170,180 (e.g., via a main tube 185 running along the arm 130), such that the air sucked into each point sensor 160 is brought back to the analyzer unit 170,180 via respective tubes 165,185 and analyzed by the analyzer unit 170,180. The analyzer unit 170,180 can be, for example, a computer with software stored thereon that is configured to receive the air and detect the concentration of a preselected analyte in the air to generate data indicated of the analyte concentration in the air. The analyzer unit can include a portion in a casing 180, which may include a fan used to pump air through the tubes 165,185 (see, e.g., FIGS. 4C-4E) and/or a computer 170 with software stored thereon. The data indicative of the analyte concentration in the air can include spatial information (e.g., the concentration in space within the field) and/or volume information (e.g., the concentration spatially and by height from the ground). For example, the point sensors 160 can be disposed at different heights along the arm 130 (e.g., two or more point sensors at different heights on the same segment or truss of the arm). The data indicative of the analyte concentration can also include a flux of the analyte concentration. The analyzer unit can be in operable communication with a display on which the data indicative of the analyte concentration in the air can be displayed. The display can be located on the center pivot or the arm or can be remote from the computer, in which case the data can be transmitted to the display wirelessly or via a wire (e.g., a buried wire). As the arm 130 moves around the center pivot 110, a full picture of analyte concentration in the entire field can be obtained, as shown in FIG. 7.

The point sensors 160 can be positioned along the arm 130 in any reasonable manner. For example, a point sensor 160 can be disposed on and/or attached to each segment of the arm (or every other segment or every third segment, etc.) or each truss of the arm (or every other truss or every third truss, etc.). The point sensors 160 can also be disposed in a non-regular manner, with some segments or trusses having one or more point sensors and others having no point sensors or less point sensors. In a preferred embodiment, the point sensors 160 are disposed at regular intervals along the arm 130 and disposed close enough to each other to generate a map of the analyte concentration with a desired granularity (e.g., one point sensors disposed on each segment or truss of the arm). Each segment or truss may have multiple point sensors at differing heights to generate a vertical (height-direction) profile of the analyte concentration.

Regardless of whether the system includes reflectors or point sensors (or both), the system can include a meteorological station in operable communication with the analyzer unit (e.g., wirelessly or wired, such as via a buried wire). The meteorological station is configured to obtain meteorological data of the air at a location over or adjacent to the field having the center pivot. The meteorological data can include wind speed, wind direction, air pressure, air temperature, and/or humidity. In a preferred embodiment, the meteorological data includes all of wind speed, wind direction, air pressure, air temperature, and humidity, allowing for accurate determination of flux of the analyte concentration.

Though the arm attached to the center pivot is referred to herein in the singular, in some embodiments a first arm can extend in a first radial direction (of the field) from the center pivot over a portion of, most of, almost all of, or an entirety of, a radius of the field, and a second arm can extend in a second radial direction (opposite from the first radial direction) from the center pivot. Each arm can have any or all of the elements discussed herein disposed thereon and/or attached thereto. In the case of two arms, two lasers for each desired wavelength can be provided, one pointing in the first radial direction and one pointing in the second radial direction.

Although center pivot systems have been discussed at length herein, systems of embodiments of the subject invention can also be used with linear move irrigation systems.

Systems and methods of embodiments of the subject invention can measure spikes in analyte concentration above the background concentration. In this way, any horizontal flux that may be present from neighboring agricultural fields (e.g., from wind blowing) can already be accounted for in the background, and spikes (and valleys) in analyte concentration compared to the background can be considered to identify hotspots (or “cold” spots) of the analyte in the field being monitored.

The analyte can be a greenhouse gas or air pollutant, such as nitrous oxide (N₂O), ammonia (NH₃), methane (CH₄), nitric oxide (NO), water vapor, carbon monoxide (CO), ozone (O₃), or carbon dioxide (CO₂). In some embodiments, the concentration in the air of more than one analyte can be detected, and each analyte may be a greenhouse gas or air pollutant (such as those listed in the previous sentence).

The systems and methods of embodiments of the subject invention can detect analyte concentration in the air with a sensitivity/precision of 500 ppb or less, such as 100 ppb or less, 50 ppb or less, 10 ppb or less, 1 ppb or less, 0.1 ppb or less, about 1 ppb, 1 ppb, about 0.1 ppb, or 1 ppb.

Embodiments of the subject invention can advantageously provide extremely accurate measurements (e.g., sensitivity of less than 10 ppb and/or precision on the ambient levels of better than one part in one thousand) of the concentration in air of one or more different analytes, such as greenhouse gases and air pollutants. This can help ensure that any emissions or concentrations can be accurately documented for purposes of reporting, regulation, marketing, and/or claims on environmental sustainability. Also, hotspots of the analyte can be identified, allowing the owner of the property/field to potentially address the hotspot and decrease air emissions. This has environmental and economic advantages (e.g., more efficient operation, lower risk of fines, recovery of lost products).

Embodiments of the subject invention can measure the concentration of greenhouse gas analytes without adding any additional infrastructure to a center pivot setup on an agricultural field. The analyzer unit and reflectors and/or point sensors can be disposed on and/or attached to the existing infrastructure of the center pivot and arm. The meteorological station can be disposed on and/or attached to the center pivot, the arm, an existing fence, or another existing post or tower. In some alternative embodiments, a new post or tower can be installed on which the meteorological station can be disposed.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

When ranges are used herein, combinations and subcombinations of ranges (e.g., any subrange within the disclosed range) and specific embodiments therein are intended to be explicitly included. When the term “about” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. A system for measuring at least one analyte in air over an agricultural field, the system comprising:
sensing equipment disposed on a center pivot and an arm attached to the center pivot; and
an analyzer unit in operable communication with the sensing equipment,
wherein the system does not require any infrastructure to be added to the center pivot or the arm.

2. The system according to claim 1, further comprising a meteorology station in operable communication with the analyzer unit, wherein the meteorological station is configured to obtain meteorological data of the air over the agricultural field, air adjacent to the agricultural field, or both,
wherein the meteorological data comprises wind speed, wind direction, air pressure, air temperature, humidity, or a combination thereof.

3. The system according to claim 1, wherein each analyte of the at least one analyte is a greenhouse gas or air pollutant.

4. The system according to claim 1, wherein the system is configured to measure the at least one analyte with a sensitivity of 100 parts per billion (ppb) or less.

5. The system according to claim 1, wherein the sensing equipment comprises:
at least one light source disposed on the center pivot; and
a plurality of reflectors disposed on the arm and configured to reflect light from the at least one light source,
wherein the plurality of reflectors are disposed close enough to each other along the arm for the analyzer unit to generate a map of a concentration of the at least one analyte with a predetermined granularity, and
wherein the sensing equipment further comprises a detector disposed on the center pivot and configured to receive signals of light reflected from the plurality of reflectors.

6. The system according to claim 5, wherein each light source of the at least one light source is configured to provide light at a predetermined wavelength for a particular analyte, and wherein the predetermined wavelength is in a range of from 2 micrometers (μm) to 30 μm.

7. The system according to claim 5, wherein the analyzer unit comprises software stored thereon that is configured to receive the signals of light reflected from the plurality of reflectors and convert them to data indicative of a concentration of the at least one analyte in the air,
wherein the data indicative of the concentration of the at least one analyte in the air comprises at least one of: spatial information of the concentration of the at least one analyte in the air; volume information of the concentration of the at least one analyte in the air; and a flux of the concentration of the at least one analyte in the.

8. The system according to claim 7, wherein the analyzer unit converts the signals via wavelength modulation spectroscopy, direct absorption spectroscopy, or both.

9. The system according to claim 5, wherein each reflector of the plurality of reflectors is a retroreflector configured to reflect mid-IR light,
wherein the retroreflector comprises:
a base substrate;
a coating layer disposed on the base substrate;
an adhesive layer disposed between the base substrate and the coating layer; and
a protective layer disposed on the coating layer.

10. The system according to claim 9, wherein the adhesive layer comprises a transition metal, and
wherein the adhesive layer has a thickness of 10,000 Angstroms or less.

11. The system according to claim 9, wherein the protective layer comprises an insulative material,
wherein the base layer comprises a thermoplastic material,
wherein the coating layer comprises a metal, and
wherein the coating layer has a thickness of 10,000 Angstroms or less.

12. The system according to claim 9, wherein the retroreflector has a total thickness of 50 millimeters (mm) or less.

13. The system according to claim 5, wherein each light source of the at least one light source is a laser.

14. The system according to claim 1, wherein the sensing equipment comprises:
a plurality of point sensors disposed on the arm and configured to suck air thereinto from the air over the agricultural field; and
a main tube connected to the analyzer unit and in fluid communication with the plurality of point sensors,
wherein the analyzer unit comprises a pumping element configured to pump air from the plurality of point sensors to the analyzer unit through the main tube,
wherein the sensing equipment further comprises a plurality of connection tubes respectively connecting the plurality of point sensors to the main tube, and wherein the plurality of point sensors are disposed close enough to each other along the arm for the analyzer unit to generate a map of a concentration of the at least one analyte with a predetermined granularity.

15. The system according to claim 14, wherein the pumping element comprises a fan.

16. The system according to claim 14, wherein the analyzer unit comprises software stored thereon that is configured to receive the air sucked into the plurality of point sensors and convert it to data indicative of a concentration of the at least one analyte in the air, and
wherein the data indicative of the concentration of the at least one analyte in the air comprises at least one of: spatial information of the concentration of the at least one analyte in the air; volume information of the concentration of the at least one analyte in the air; and a flux of the concentration of the at least one analyte in the air.

17. The system according to claim 16, wherein the analyzer unit converts the air via wavelength modulation spectroscopy, direct absorption spectroscopy, or both.

18. The system according to claim 14, wherein the plurality of point sensors are disposed along the arm at a plurality of different heights, as measured from a ground of the agricultural field.

19. The system according to claim 1, wherein the analyzer unit is configured to measure a concentration in the air over the agricultural field of each analyte of the at least one analyte based on spikes and valleys compared to a respective background concentration of each analyte of the at least one analyte.

20. A method for measuring at least one analyte in air over an agricultural field, the method comprising:
providing the system according to claim 1;
moving the arm around the center pivot;
using the sensing equipment to obtain air or signals of reflected light; and
using the analyzer unit to convert the air or the signals of reflected light to data indicative of a concentration of the at least one analyte in the air.
